(19) United States
(12) Patent Application Publication
(10) Pub. No.: US 2012/0108641 A1
(43) Pub. Date: May 3, 2012

(54) ANTITUMOR COMBINATION INCLUDING AVE8062 AND SORAFENIB

(75) Inventors: Brigitte DEMERS, Paris (FR); Patricia VRIGNAUD, Combs La Ville (FR)

(73) Assignee: SANOFI, Paris (FR)

(21) Appl. No.: 13/288,236

(22) Filed: Nov. 3, 2011

Related U.S. Application Data
(63) Continuation of application No. PCT/FR2010/050874, filed on May 6, 2010.

(30) Foreign Application Priority Data
May 7, 2009 (FR) 09/02210

(51) Int. Cl. A61K 31/44; A61P 35/00

(52) U.S. Cl. 514/350

(57) ABSTRACT

Combinations comprising AVE8062 and sorafenib are provided which are effective in the treatment of cancers, more particularly of solid tumours.

### CROSS-REFERENCE TO RELATED APPLICATIONS

This application is a continuation of International Application No. PCT/FR2010/050874, filed May 6, 2010, which claims the benefit of priority of French patent application Ser. No. 09/02210, filed May 7, 2009, both of which are incorporated herein by reference.

The present invention relates to an antitumour combination that combines AVE8062 and sorafenib that is effective in the treatment of cancers, more particularly of solid tumours.

### BACKGROUND

WO 2007/077309 describes the combination between the antivascular agent AVE8062 (or VDA, Vascular Disrupting Agent) and the antiangiogenic agent VEGF Trap.

WO 99910779 describes the AVE8062/platinum salt combination.

WO 2004/037258 describes the combination of AVE8062 with various antitumour agents chosen from taxanes (taxol, taxotere), alkylating agents (cyclophosphamide, isosfamide, etc.), antimetabolites (5-FU, cytarabine, etc.), epidophylloptoxin, antibiotics (doxorubicin, etc.), and vinca alkaloids.

EP 1407784 describes the AVE8062/dexamethasone combination.

On the website www.clinicaltrials.gov, the patient recruitment phase for the Phase I study of the combretastatin CA4P/Avastin combination (“Safety study of increasing doses of combretastatin in combination with Bevacizumab (Avastin) in patients with advanced solid tumours”) is described. It is specified that the patients excluded are those who have already undergone a treatment based on a VEGF or VEGFR inhibitor such as sorafenib or sutent (“exclusion criteria: prior therapy with CA4P or bevacizumab, or other agents which target vascular endothelial growth factor (VEGF) or VEGFR signaling such as Sorafenib and Sutent”).

In the file on Nexavar available on the website of the EMEA (http://www.emea.europa.eu/humandocs/PDFs/EPAR/nexavar/H-690-PI-fr.pdf), it is indicated that Nexavar® (sorafenib tosylate) may be combined with various anticancer agents such as gemcitabine, oxaliplatin, doxorubicin, irinotecan or docetaxel.

### SUMMARY

The invention relates to an antitumour pharmaceutical combination comprising AVE8062 of formula:

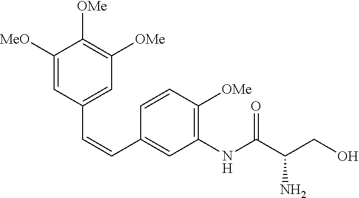

and sorafenib of formula:

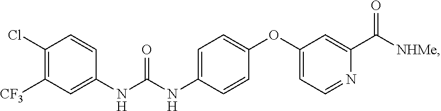

these two antitumour agents possibly being in the base form or in the form of a salt of a pharmaceutically acceptable acid. The combination comprises an effective amount of AVE8062 and an effective amount of sorafenib.

The combination is intended to be administered to a patient during a cycle comprising an administration of AVE8062 that marks the beginning of said cycle and several administrations of sorafenib, the combination being staggered over time and not concomitant, the AVE8062 being administered before the very first administration of sorafenib. The AVE8062 may be administered the same day as the sorafenib with a time delay of 1 to 4 hours before the very first administration of sorafenib. The AVE8062 may also be administered the day before the very first administration of sorafenib, more particularly with a time delay of at least 24 hours.

The cycle is repeated, the interval between two administrations of AVE8062 ranging from 1 to 4 weeks.

The invention also relates to the use of AVE8062 and sorafenib for the preparation of the antitumour combination described above.

### DETAILED DESCRIPTION

### Definitions

pharmaceutically acceptable acid: organic or inorganic acid having a low toxicity (see “Pharmaceutical salts” J. Pharm. Sci. 1977, 66, 1-19);
effective amount: amount of a pharmaceutical compound that produces an effect on the treated tumour.

Regarding AVE8062, this belongs to the family of combretastatins and has the formula:

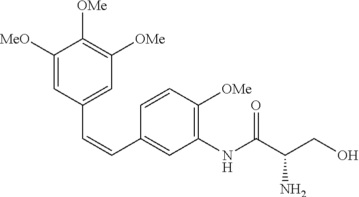

(it is the Z isomer)

It is an antivascular agent (or VDA, Vascular Disrupting Agent). It has the chemical name: (Z)—N-[2-methoxy-5-[2-(3,4,5-trimethoxyphenyl)vinyl]phenyl]-L-serinamide. This compound, which is described in EP 731085 B1, may be prepared according to the method described in WO 03/084919. AVE8062 may be administered in base form (cf. above formula) or in the form of a salt of a pharmaceutically acceptable acid, for example in the form of the hydrochloride, represented below:

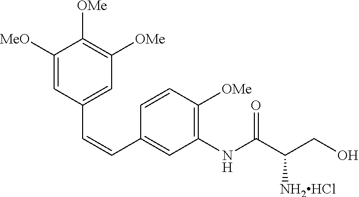

Once administered, AVE8062 releases in vivo the active metabolite (Z)-1-(3-amino-4-methoxyphenyl)-2-(3,4,5-trimethoxyphenyl)ethene, which has the formula:

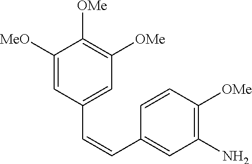

It is therefore also possible to substitute, for AVE8062, another combretastatin of formula:

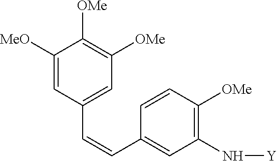

in base form or in the form of a salt of a pharmaceutically acceptable acid, in which Y represents an amino acid, which releases in vivo this metabolite.

Regarding sorafenib, this is sold by Bayer HealthCare under the trademark Nexavar®. Sorafenib is a multikinase inhibitor that targets VEGF and BRAF receptors which has the chemical formula:

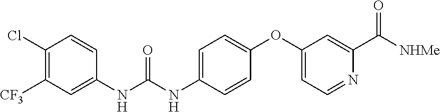

and has the chemical name: 4-[4-[[4-chloro-3-(trifluoromethyl)phenyl]carbamoylamino]phenoxy]-N-methylpyridine-2-carboxamide. It is an antiangiogenic agent. This compound is described in WO 00/42012 and WO 00/41698. Sorafenib may be administered in base form (cf. formula above) or in the form of a salt of a pharmaceutically acceptable acid, for example in tosylate form.

Regarding the combination, this consists in combining, in the form of two separate pharmaceutical preparations, AVE8062 and sorafenib.

The combination is administered repeatedly in a course of several cycles according to a protocol that depends on the nature and on the stage of the cancer to be treated and also on the patient to be treated (age, weight, previous treatment(s), etc.). Each cycle begins with the administration of AVE8062 and comprises, in addition to this, several administrations of sorafenib (one cycle is therefore characterized by an administration of AVE8062 that marks the beginning of said cycle and several administrations of sorafenib). The AVE8062 is administered to a patient in an intermittent pattern with an interval between two administrations (duration of one cycle) which may range from 1 to 4 weeks, for example 3 weeks (comment: in the case of tests on mice, the administration interval of AVE8062 was 4 or 5 days). Sorafenib may itself be administered to a patient in a daily pattern over a certain duration of the cycle. Sorafenib may optionally be administered up to the end of one cycle.

The mode of administration may be the parenteral route and/or oral route and depends on the galenic form use for the antitumour agent. By the parenteral route, the antitumour agent may be administered intravenously as a bolus or prepared in an intravenous infusion bag, with pharmaceutically acceptable vectors, by various methods known to a person skilled in the art. According to one particular mode, the AVE8062 is administered parenterally, such as via intravenous administration, as a bolus or via infusion, and the sorafenib is administered orally.

One galenic form of AVE8062 suitable for parenteral administration is that where the AVE8062 is in solution in water. One galenic form of sorafenib suitable for oral administration is, for example, that sold under the trademark Nexavar® in the form of tablets containing 274 mg of sorafenib in sorafenib tosylate form (equivalent to 200 mg of active principle).

The doses of AVE8062 and of sorafenib administered each time to a patient depend on various parameters such as the nature and the stage of the cancer to be treated and also on the patient to be treated (age, weight, previous treatment(s), etc.). The AVE8062 may be administered at a tolerated dose between 5 and 100, 5 and 60, 10 and 50, 20 and 42 or 20 and 40 mg/m² (weight/body surface area, dose defined for each administration). The sorafenib may itself be administered at a tolerated dose between 200 and 600 mg, or 300 and 500 mg (dose defined for each administration). The sorafenib may be taken two times a day at a dose of active principle of 200 mg, which corresponds to a daily dose of 400 mg. Furthermore, according to the product instructions, it is recommended to take this product at least one hour before or two hours after a meal.

The combination is effective in the treatment of cancers, more particularly of solid tumours in general, more particularly of a sarcoma, lung, ovarian, kidney or liver cancers.

It has been observed that a better efficacy in the treatment of the tumour is obtained when, in the course of one cycle, the administration of the two antitumour agents is staggered over time and is not concomitant, the AVE8062 being administered before the very first administration of the sorafenib.

According to one particular mode, the AVE8062 is administered the same day and with a time delay of 1 to 4 hours before the very first administration of sorafenib. A cycle example: day D1: infusion of AVE8062 and, 1 to 4 hours after the infusion, sorafenib is taken orally (e.g. in the form of two doses of sorafenib); day D2 to D14: sorafenib is taken orally (e.g. in the form of two doses of sorafenib), then the cycle is repeated at D1+3 weeks.

According to another particular mode, the AVE8062 is administered the day before the very first administration of sorafenib. More particularly, the time delay between the administration of the AVE8062 and the very first administration of sorafenib is at least 24 hours. Cycle example: day D1: infusion of AVE8062; day D2 after a time delay of at least 24 hours: sorafenib is taken orally (e.g. in the form of two doses of sorafenib); day D3 to D14: sorafenib is taken orally (e.g. in the form of two doses of sorafenib), then the cycle is repeated at D1+3 weeks.

The efficacy of a combination can be demonstrated by determining its therapeutic synergy. A combination manifests therapeutic synergy if it is therapeutically superior to the best agent used alone at its optimum dose (T. H. Corbett et al., Cancer Treatment Reports 1982, 66, 1187). The efficacy of a combination can also be demonstrated by comparing the maximum tolerated dose of the combination with the maximum tolerated dose of each of the separate constituents in the study in question. This efficacy can be quantified by the log₁₀ cell kill, which is determined by the following formula:

log₁₀ cell kill=T−C (days)/3.32×T_{d}

in which T−C represents the tumour growth delay, which is the mean time, in days, required by the treatment-group tumours (T) to reach a predetermined value (1 g for example) and for the control-group tumours (C) to reach the same value, and T_{d} represents the time, in days, necessary for the volume of the control-group tumours to double during the exponential phase of tumour growth (T. H. Corbett et al. Cancer, 1977, 40, 2660-2680; F. M. Schabel et al., Cancer Drug Development, Part B, Methods in Cancer Research 1979, 17, 3-51, New York, Academic Press Inc.). A product is considered to be active if the log₁₀ cell kill is greater than or equal to 0.7. A product is considered to be highly active if the log₁₀ is greater than 2.8. When the treatment time is at least equal to 10 days, and/or is different between the two agents evaluated in the combination, it is possible to calculate the net log cell kill:

net log₁₀ cell kill=(T−C in days)−(treatment time in days)/3.32×T_{d}.

The activity in this case is acknowledged for a net log cell kill that is positive (>0). A cytostatic activity corresponds to a net log cell kill of 0, that is to say that the treatment time is equal to the duration of the antitumour effect.

The combination, used at its own maximum tolerated dose, in which each of the constituents is present at a dose that generally does not exceed its maximum tolerated dose, will show therapeutic synergy when the log₁₀ cell kill is at least 1 log₁₀ greater than the value of the log₁₀ cell kill of the best constituent when the latter is administered alone.

### Examples

### Antitumor Effect and Tests

The efficacy of the combinations on solid tumours can be determined experimentally in the following way: the animals subjected to the experiment are female SCID mice which are bilaterally grafted subcutaneously with 30 to 60 mg of a fragment of NCI-H460 (ATCC#HTB-177) human non-small cell lung tumour at day 0. In the case of an early tumour treatment, the implanted animals are distributed randomly in various groups which are, or are not (controls), intended to receive the treatment(s). Where it is a question of treatment of advanced tumours, the animals bearing tumours that have reached a predefined tumour size greater than 150 mg are distributed in the various treatment and control groups in such a way that the tumour size range is comparable from one group to the other. The animals which do not bear tumours can also be subjected to the same treatments as the animals bearing tumours, so that it is possible to dissociate the toxic effect from the specific effect on the tumour. Generally, the chemotherapy begins from 3 to 22 days after the graft, depending on the type of tumour and the desired tumour size. The animals are observed and weighed every day. A dose which induces a weight loss of 20% or more at the lowest point (average of the group) or a mortality of 10% or more is considered to be toxic. The tumour activity is evaluated at the highest non-toxic dose, or at the highest dose tested, within the context of a non-cytotoxic agent.

The tumours are measured 2 or 3 times a week until the tumour reaches approximately 2 g or until the animal dies, if this occurs before the tumour reaches 2 g. The animals are autopsied when they are sacrificed.

The antitumor activity is determined in accordance with various recorded parameters, such as the dose (mg/kg), the method of administration, the administration time, the toxicity and the log₁₀ cell kill, which depends on the tumour growth delay and also on the tumour doubling time.

Within the context of the following studies, the AVE8062, in hydrochloride form, is formulated in water with 0.9% NaCl. The sorafenib is formulated with 12.5% of ethanol, 12.5% of polysorbate 80 and 75% of 5% glucose in water.

Study 1: Sorafenib Administered Simultaneously with AVE8062 (Table I)

The AVE8062 was administered intravenously on days 9 and 13 post tumour implantation. The sorafenib was administered orally from day 9 to day 24. When the two agents were administered in combination, the same schedules were used as for the agents alone, the combination of the two agents having been carried out simultaneously on days 9 and 13.

The tumour doubling time was two days.

The median tumour weight at the start of the treatments was 219 to 234 mg, the control having reached a tumour weight of 1000 mg, 12.8 days after the tumour grafting.

The highest evaluated dose (HED) of AVE8062 is 58 mg/kg per injection, i.e. a total dose of 116 mg/kg. At this dose, AVE8062 is active with 0.9 log₁₀ cell kill (log cell kill), 1/6 partial regression (PR=50% regression of the initial tumour size) being obtained at this dose.

The sorafenib, at its highest dose tested (HDT) of 62 mg/kg per administration, i.e. a total dose of 992 mg/kg, is also active with 2.3 log cell kill. However, the sorafenib did not have a cytostatic activity at this dose (−0.1 net log cell kill), the tumour having escaped treatment.

The highest non-toxic dose (HNTD) of the combination was determined at the dose of 36 mg/kg per administration of AVE8062 combined with that of 62 mg/kg per administration of sorafenib, higher doses of the combination having been found to be toxic. At this HNTD, the combination is active with 2.4 log cell kill, and 0.0 net log cell kill. However, no partial regression was observed at this dose. Lower doses of the combination are also active (2.2 to 2.5 log cell kill), without inducing tumour regression either.

In conclusion, the concomitant administration of AVE8062 and of sorafenib is active, maintaining at least the therapeutic gain of each of the two agents alone. Furthermore, it has been possible to observe that this activity is maintained at several dose levels only for the combination.

Study 2: Sorafenib Administered 1 h after AVE8062 in the Combination (Table II)

The AVE8062 was administered intravenously on days 10 and 14 post tumour implantation. The sorafenib was administered orally from day 10 to day 14. The two agents were administered in combination, according to the same schedules as those used for the agents alone, but the administration of sorafenib having been staggered one hour after the administration of the AVE8062.

The tumour doubling time was 1.6 days.

The median tumour weight at the start of the treatments was 431 to 458 mg, the control having reached a tumour weight of 1500 mg, 13.2 days after tumour grafting.

The 2 highest doses of AVE8062 were toxic and the highest non-toxic dose (HNTD) is 22.3 mg/kg per injection, i.e. a total dose of 44.6 mg/kg. At this dose, AVE8062 is active with 1.1 log₁₀ cell kill (log cell kill), without inducing tumour regression.

Sorafenib, at its highest dose tested (HDT) of 100 mg/kg per administration, i.e. a total dose of 447.4 mg/kg, is also active with 1.1 log cell kill.

The HNTD of the combination was determined at the dose of 58 mg/kg per administration of AVE8062 combined with that of 38.4 mg/kg per administration of sorafenib, the higher doses of the combination having been found to be toxic. At this HNTD, the combination is active with 2.1 log cell kill, i.e. 1 log cell kill more than the agents alone, 1.1 log cell kill for each). Furthermore, 50% (3/6) of partial regressions (PR=50% regression of the initial tumour size) was obtained at this dose. Five lower doses of the combination are also active, with a log cell kill of 1.9 to 1.5, and inducing PR at 4 dose levels.

In conclusion, the combination of AVE8062 with sorafenib administered 1 h later, induces more tumour regressions than each of the agents alone, a therapeutic synergy being observed at the HNTD.

Study 3: Sorafenib Administered 24 h after AVE8062 in the Combination (Table III)

The AVE8062 was administered intravenously on days 9 and 14 post implantation of the NCI-H460 pulmonary tumour in female SCID mice. The sorafenib was administered orally from day 9 to day 20. When the 2 agents were administered in combination, the same schedules were used as for the agents alone, but the administrations of sorafenib were started 24 hours after that of AVE8062.

The tumour doubling time was 1.5 days.

The median tumour weight at the start of the treatments was 217 to 235 mg, the control having reached a tumour weight of 1000 mg, 13.6 hours after tumour grafting.

The highest non-toxic dose (HNTD) of the AVE8062 is 36 mg/kg per injection, i.e. a total dose of 72 mg/kg. At this dose, AVE8062 is active with 1.7 log₁₀ cell kill (log cell kill) without inducing tumour regression.

Sorafenib, at its highest dose tested (HDT) of 100 mg/kg per administration, i.e. a total dose of 1213.3 mg/kg, is also active with 2.4 log cell kill. However, sorafenib did not have a cytostatic activity at this dose (−0.4 net log cell kill), the tumour having escaped treatment.

The HNTD of the combination was determined at the dose of 36 mg/kg per administration of AVE8062 combined with that of 100 mg/kg per administration of sorafenib, the higher doses of the combination having been found to be toxic. At this HNTD, the combination is highly active with 3.1 log cell kill, and 0.3 net log cell kill. Furthermore, 50% (3/6) of partial regressions (PR=50% regression of the initial tumour size) and 16% (1/6) of complete regression (CR=regression below the palpable limit of 63 mg) were obtained at this dose. Lower doses of the combination are also active (2.6 to 3 log cell kill), and induce PR at 5 dose levels and CR at 2 dose levels.

In conclusion, this combination, which uses a sequence during which sorafenib is administered after AVE8062, induces complete and/or partial tumour regressions, which is not observed for the agents alone. These regressions, in combination, are observed at several dose levels. The log₁₀ cell kill in combination is systematically higher than that observed in monotherapy.

In conclusion, for these 3 studies, staggering the administration of sorafenib by at least 1 hour after the administration of AVE8062, gives a therapeutic gain compared to the administration of the two antitumour agents when they are administered alone. Expanding this interval up to at least 24 hours increases this therapeutic advantage.

TABLE I
Evaluation of AVE8062 in combination with sorafenib (simultaneously) on female
SCID mice bearing the NCI-H460 human tumour.

| | Dosage in | | | | | | |
|---|---|---|---|---|---|---|---|
| Agent | mg/kg per | Total dose | log cell | net log | | | |
| (schedule) | injection | in mg/kg | kill | cell kill | PR | CR | Observations |
| AVE8062 | 58.0 | 116.0 | 0.9 | 0.1 | 1/6 | 0/6 | HED - active |
| (9, 13) | 36.0 | 72.0 | 0.8 | 0.0 | 1/6 | 0/6 | Active |
| | 22.3 | 44.6 | 0.6 | −0.1 | 0/6 | 0/6 | Inactive |
| Sorafenib | 62.0 | 992.0 | 2.3 | −0.1 | 0/6 | 0/6 | HDT - no cytostatic activity |
| (9-24) | 38.4 | 614.4 | 1.5 | −0.9 | 0/6 | 0/6 | No cytostatic activity |
| AVE8062/sorafenib | 36.0/62.0 | 72.0/992.0 | 2.4 | 0.0 | 0/6 | 0/6 | HNTD - active |
| (9, 13) (9-24) | 22.3/62.0 | 44.6/992.0 | 2.4 | 0.0 | 0/6 | 0/6 | Active |
| | 36.0/38.4 | 72.0/614.4 | 2.4 | 0.0 | 0/6 | 0/6 | Active |
| | 13.8/62.0 | 27.6/992.0 | 2.4 | 0.0 | 0/6 | 0/6 | Active |
| | 8.6/62.0 | 17.2/992.0 | 2.4 | 0.0 | 0/6 | 0/6 | Active |
| | 22.3/38.4 | 44.6/614.4 | 2.5 | 0.0 | 0/6 | 0/6 | Active |
| | 13.8/38.4 | 27.6/614.4 | 2.5 | 0.0 | 0/6 | 0/6 | Active |
| | 8.6/38.4 | 8.6/614.4 | 2.2 | −0.3 | 0/6 | 0/6 | No cytostatic activity |

Tumour doubling time = 2 days. Median tumour weight at the start of the treatments = 219-234 mg. Median time to reach 1000 mg in the control = 12.8 days. Treatment time: sorafenib = 16 days, AVE8062 = 5 days.
Formulation: AVE8062 in water with 0.9% NaCl; sorafenib = 12.5% ethanol, 12.5% PS80, 75% of 5% glucose in water.
Abbreviations:
HED = highest evaluated dose,
HNTD = highest non-toxic dose,
HDT = highest dose tested,
PR = partial regressions,
CR = complete regressions.

TABLE II
Evaluation of AVE8062 in combination with sorafenib (1 h after AVE8062) on female SCID mice bearing the NCI-
H460 human tumour.

| Agent, route | | | | % BWC | | | | | |
|---|---|---|---|---|---|---|---|---|---|
| Dose in mg/kg/admin | | | Mortality | (day of | T − C | log | | | |
| mg/kg) | | Schedule in | (day of | lowest | in | cell | Regressions | | |
| AVE8062. i.v. | sorafenib. p.o. | days | death) | point) | days | kill | partial | complete | Observations |
| 58.0 (58.0) | | 10 | 3/5 (2 d12, 13) | −6.9 (11) | — | — | — | — | Toxic |
| 36.0 (36.0) | | | 1/5 (12) | −3.9 (11) | — | — | — | — | Toxic |
| 22.3 (44.6) | | 10, 14 | 0/5 | −3.2 (21) | 5.8 | 1.1 | 0/5 | 0/5 | HNTD - active |
| | 100.0 (447.4) | 10-14 | 0/6 | −4.8 (17) | 6.1 | 1.1 | 0/6 | 0/6 | HDT - Active |
| | 62.0 (277.6) | | 0/6 | −7.1 (16) | 4.4 | 0.8 | 0/6 | 0/6 | Modest activity |
| | 38.4 (171.9) | | 0/6 | −4.2 (15) | 4.5 | 0.8 | 0/6 | 0/6 | Active |
| 58.0 (116.0) | 100.0 (447.4) | 10, 14/10-14 | 1/6 (15) | −13.6 (16) | — | — | — | — | Toxic |
| | 62.0 (277.6) | | 1/6 (15) | −15.9 (15) | — | — | — | — | Toxic |
| | 38.4 (171.9) | | 0/6 | −10.3 (15) | 10.9 | 2.1 | 3/6 | 0/6 | HNTD - active |
| 36.0 (72.0) | 100.0 (447.4) | | 1/6 (16) | −12.9 (15) | — | — | — | — | Toxic |
| | 62.0 (277.6) | | 0/6 | −9.5 (15) | 10.2 | 1.9 | 1/6 | 0/6 | Active |
| | 38.4 (171.9) | | 0/6 | −8.7 (15) | 9.5 | 1.8 | 2/6 | 0/6 | Active |
| 22.3 (44.6) | 100.0 (447.4) | | 0/6 | −7.7 (15) | 9.4 | 1.8 | 1/6 | 0/6 | Active |
| | 62.0 (277.6) | | 0/6 | −5.6 (15) | 8.0 | 1.5 | 1/6 | 0/6 | Active |
| | 38.4 (171.9) | | 0/6 | −6.2 (15) | 8.1 | 1.5 | 0/6 | 0/6 | Active |

Tumour doubling time = 1.6 days. Median tumour weight at the start of the treatments = 431-458 mg. Median time to reach 1500 mg in the control = 13.2 days.
Formulation: AVE8062 with 0.9% NaCl; sorafenib = 12.5% ethanol, 12.5% PS80, 75% of 5% glucose in water.
Abbreviations:
HNTD = highest non-toxic dose,
HDT = highest dose tested,
BWC = body weight change.

TABLE III
Evaluation of AVE8062 in combination with sorafenib (24 h after AVE8062) on female SCID
mice bearing the NCI-H460 human tumour.

| | | | | net | | | |
|---|---|---|---|---|---|---|---|
| | Dosage in | | gross | log | | | |
| | mg/kg per | Toatl dose in | log cell | cell | | | |
| Agent | injection | mg/kg | kill | kill | PR | CR | Observations |
| AVE8062 | 36.0 | 72.0 | 1.7 | 0.3 | 0/6 | 0/5 | HNTD - active |
| (9, 14) | 22.3 | 44.6 | 1.7 | 0.3 | 0/6 | 0/6 | Active |
| Sorafenib | 100.0 | 1213.3 | 2.4 | −0.4 | 0/6 | 0/6 | HDT - no cytostasis |
| (9-20) | 62.0 | 752.2 | 2.3 | −0.5 | 0/6 | 0/6 | No cytostasis |
| AVE8062/sorafenib | 58.0/100.0 | 116.0/1101.3 | 3.3 | 0.5 | 5/6 | 3/6 | HED - highly active |
| (9, 14) (9-20) | 36.0/100.0 | 72.0/1101.3 | 3.1 | 0.3 | 3/6 | 1/6 | HNTD - highly active |
| | 36.0/62.0 | 72.0/682.8 | 3.0 | 0.2 | 4/6 | 2/6 | Highly active |
| | 22.3/100.0 | 44.6/1101.3 | 2.7 | −0.1 | 2/6 | 0/6 | No cytostasis |
| | 13.8/100.0 | 27.6/1101.3 | 2.6 | −0.2 | 0/6 | 0/6 | No cytostasis |
| | 22.3/62.0 | 44.6/682.8 | 3.0 | 0.2 | 5/6 | 2/6 | Highly active |
| | 8.6/100.0 | 17.2/1101.3 | 2.8 | 0.0 | 0/6 | 0/6 | Active |
| | 13.8/62.0 | 27.6/682.8 | 2.9 | 0.1 | 1/6 | 0/6 | Highly active |
| | 8.6/62.0 | 8.6/682.8 | 2.8 | 0.0 | 2/6 | 1/6 | Active |

Tumour doubling time = 1.5 days. Median tumour weight at the start of the treatments = 217-235 mg. Median time to reach 1000 mg in the control = 13.6 days. Treatment time: Combination and sorafenib alone = 12 days, AVE8062 alone = 5 days.
Formulation: AVE8062 with 0.9% NaCl; sorafenib = 12.5% ethanol, 12.5% PS80, 75% of 5% glucose in water.
Abbreviations:
HNTD = highest non-toxic dose,
HDT = highest dose tested,
PR = partial regressions,
CR = complete regressions.

We claim:

1. A composition comprising AVE8062 of formula: 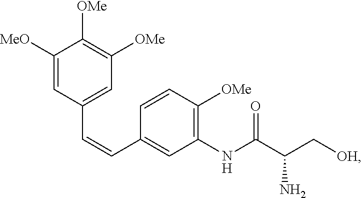
or a pharmaceutically acceptable salt or base thereof, and sorafenib of formula: 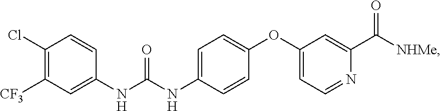
or a pharmaceutically acceptable salt or base thereof.

2. The composition of claim 1, comprising an effective amount of AVE8062 and an effective amount of sorafenib.

3. The composition of claim 1, wherein the AVE8062 is in hydrochloride form and/or the sorafenib is in tosylate form.

4. The composition of claim 1, wherein the AVE8062 and the sorafenib are in amounts that produce a synergistic effect in reducing tumor volume in a patient when said composition is administered to a patient.

5. A method of treating a patient with cancer, comprising administering to said patient an effective amount of AVE8062 of formula: 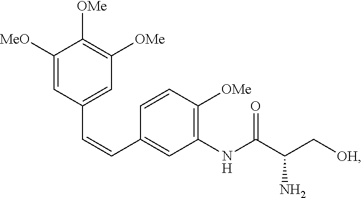
or a pharmaceutically acceptable salt or base thereof, in combination with sorafenib of formula: 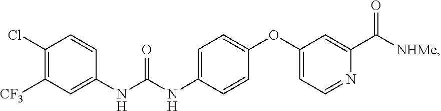
or a pharmaceutically acceptable salt or base thereof.

6. The method of claim 5, wherein the effective amount achieves a synergistic effect in reducing tumor volume in said patient.

7. The method of claim 5, wherein the method comprises at least one cycle in which the administration of the AVE8062, or pharmaceutically acceptable salt or base thereof, is followed by several administrations of the sorafenib, or pharmaceutically acceptable salt or base thereof.

8. The method of claim 7, wherein the first of said several administrations of sorafenib, or pharmaceutically acceptable salt or base thereof, is administered one to four hours after the administration of the AVE8062, or pharmaceutically acceptable salt or base thereof.

9. The method of claim 7, wherein the first of said several administrations of sorafenib, or pharmaceutically acceptable salt or base thereof, is administered at least 24 hours after the administration of the AVE8062, or pharmaceutically acceptable salt or base thereof.

10. The method of claim 7, wherein the method comprises multiple cycles and the interval between the cycles is between one and four weeks.

11. The method of claim 5, wherein the AVE8062, or pharmaceutically acceptable salt or base thereof, is administered parenterally and the sorafenib, or pharmaceutically acceptable salt or base thereof, is administered orally.

12. The method of claim 5, wherein the cancer is a solid tumor.

13. The method of claim 5, wherein the cancer is selected from the group consisting of sarcoma, lung cancer, ovarian cancer, kidney cancer and liver cancer.
